SEQUENCE LISTING (20 sequences)

SEQ ID NO: 1 - protein, 297 aa, Canis familiaris
MTTPRNSMSGTLPVDPMKSPTAMYPVQKIIPKRMPSVVGPTQNFFMRESKTLGAVQIMNGLFHIALGSLLMIHTDVCAPICITMWYPLWGGIMFIISGSLLAAADKNPRKSLVKGKMIMNSLSLFAAISGIIFLIMDIFNITISHFFKMENLNLIKAPMPYVDIHNCDPANPSEKNSLSIQYCGSIRSVFLGVFAVMLIFAFFQKLVTAGIVENEWKKLCSKPKSDVVVLLAAEEKKEQPIETTEEMVELTEIIASQPKKEEDIEIPVQEEEGELEINFAEPPQEQESSPIENDSIP

SEQ ID NO: 2 - protein, 44 aa, Canis familiaris
TISHFFKMENLNLIKAPMPYVDIHNCDPANPSEKNSLSIQYCGS

SEQ ID NO: 3 - DNA, 1238 bp, Canis familiaris
atcagccactcgccctaaggccacagacactcaggagttcagagggtgagatgacaacacccagaaattcaatgagtggaaccctcccggtagatcctatgaaaagccctactgccatgtatcctgttcaaaaaataattcccaaaaggatgccttcagtggtgggccctacacaaaacttcttcatgagggaatctaagacactgggggctgtccagattatgaatgggctcttccacattgccctaggcagcctcctgatgattcacacggatgtctgtgcgcccatctgtataactatgtggtaccctctctggggaggcattatgttcatcatttctggatcactcctggcagcagcggacaaaaaccccaggaagagtttggtcaaaggaaaaatgataatgaactcattgagcctctttgctgccatttctggaataatttttttgatcatggacatatttaatattaccatttcccatttttttaaaatggagaatttgaatcttattaaagctcccatgccatatgttgacatacacaactgtgacccagctaacccctctgagaaaaactctttatctatacaatattgtggcagcatacgatctgttttcttgggcgtttttgctgtgatgctgatctttgccttcttccagaaacttgtgacagctggcattgttgagaatgaatggaaaaaactgtgctctaaacctaaatctgatgtagttgttctgttagctgctgaagaaaaaaaagaacagccgattgaaacaacagaagaaatggttgagctgactgaaatagcttcccaaccaaagaaagaagaagacattgaaattattccagtccaagaagaagaaggggaactggaaataaactttgcagaacctccccaggagcaggaatcttcaccaatagaaaacgacagcatcccttaagtaacgtttttctttctgtttccttttcttaggcgttagtgttcacagctttcaagagacatatccacccctgtttcctgaggccccctgcaggtgggcctcctccatgtgtctctctggcctttgcatggagtgaccacagctcgcttgcgctagctcgctctctttctctcatgcagaggatgcagccattgcaggaggctaagtcgggcagcttatttacattacagcaaggcagactgtaatttctcactaaacttttccctggataaagcttaaaaaaaaaaaaaaaaa

SEQ ID NO: 4 - RNA, 1238 nt, Canis familiaris
aucagccacucgcccuaaggccacagacacucaggaguucagagggugagaugacaacacccagaaauucaaugaguggaacccucccgguagauccuaugaaaagcccuacugccauguauccuguucaaaaaauaauucccaaaaggaugccuucaguggugggcccuacacaaaacuucuucaugagggaaucuaagacacugggggcuguccagauuaugaaugggcucuuccacauugcccuaggcagccuccugaugauucacacggaugucugugcgcccaucuguauaacuaugugguacccucucuggggaggcauuauguucaucauuucuggaucacuccuggcagcagcggacaaaaaccccaggaagaguuuggucaaaggaaaaaugauaaugaacucauugagccucuuugcugccauuucuggaauaauuuuuuugaucauggacauauuuaauauuaccauuucccauuuuuuuaaaauggagaauuugaaucuuauuaaagcucccaugccauauguugacauacacaacugugacccagcuaaccccucugagaaaaacucuuuaucuauacaauauuguggcagcauacgaucuguuuucuugggcguuuuugcugugaugcugaucuuugccuucuuccagaaacuugugacagcuggcauuguugagaaugaauggaaaaaacugugcucuaaaccuaaaucugauguaguuguucuguuagcugcugaagaaaaaaaagaacagccgauugaaacaacagaagaaaugguugagcugacugaaauagcuucccaaccaaagaaagaagaagacauugaaauuauuccaguccaagaagaagaaggggaacuggaaauaaacuuugcagaaccuccccaggagcaggaaucuucaccaauagaaaacgacagcaucccuuaaguaacguuuuucuuucuguuuccuuuucuuaggcguuaguguucacagcuuucaagagacauauccaccccuguuuccugaggcccccugcaggugggccuccuccaugugucucucuggccuuugcauggagugaccacagcucgcuugcgcuagcucgcucucuuucucucaugcagaggaugcagccauugcaggaggcuaagucgggcagcuuauuuacauuacagcaaggcagacuguaauuucucacuaaacuuuucccuggauaaagcuuaaaaaaaaaaaaaaaaa

SEQ ID NO: 5 - DNA, 132 bp, Canis familiaris
accatttcccatttttttaaaatggagaatttgaatcttattaaagctcccatgccatatgttgacatacacaactgtgacccagctaacccctctgagaaaaactctttatctatacaatattgtggcagc

SEQ ID NO: 6 - DNA, 43 bp, Artificial Sequence
  Description of Artificial Sequence Synthetic oligonucleotide
agagagagagagaactagtctcgagtttttttttttttttttt

SEQ ID NO: 7 - DNA, 23 bp, Artificial Sequence
  Description of Artificial Sequence Synthetic oligonucleotide
ctctttgctgccatttctggaat

SEQ ID NO: 8 - DNA, 23 bp, Artificial Sequence
  Description of Artificial Sequence Synthetic oligonucleotide
tggaagaaggcaaagatcagcat

SEQ ID NO: 9 - DNA, 18 bp, Artificial Sequence
  Description of Artificial Sequence Synthetic oligonucleotide
tgtaaaacgacggccagt

SEQ ID NO: 10 - DNA, 17 bp, Artificial Sequence
  Description of Artificial Sequence Synthetic oligonucleotide
caggaaacagctatgac

SEQ ID NO: 11 - DNA, 37 bp, Artificial Sequence
  Description of Artificial Sequence Synthetic oligonucleotide
ggccacgcgtcgactagtacttttttttttttttttt

SEQ ID NO: 12 - DNA, 23 bp, Artificial Sequence
  Description of Artificial Sequence Synthetic oligonucleotide
ctctttgctgccatttctggaat

SEQ ID NO: 13 - DNA, 32 bp, Artificial Sequence
  Description of Combined DNA/RNA Molecule Synthetic oligonucleotide
cuacuacuacuaggccacgcgtcgactagtac

SEQ ID NO: 14 - DNA, 20 bp, Artificial Sequence
  Description of Artificial Sequence Synthetic oligonucleotide
gtgatgctgatctttgcctt

SEQ ID NO: 15 - DNA, 20 bp, Artificial Sequence
  Description of Artificial Sequence Synthetic oligonucleotide
ctggaagaaggcagagatca

SEQ ID NO: 16 - DNA, 23 bp, Artificial Sequence
  Description of Combined DNA/RNA Molecule Synthetic oligonucleotide
tggaagaaggcaaagatcagcat

SEQ ID NO: 17 - DNA, 48 bp, Artificial Sequence
  Description of Combined DNA/RNA Molecule Synthetic oligonucleotide
cuacuacuacuaggccacgcgtcgactagtacgggnngggnngggnng

SEQ ID NO: 18 - DNA, 19 bp, Artificial Sequence
  Description of Artificial Sequence Synthetic oligonucleotide
ccagaaatggcagcaaaga

SEQ ID NO: 19 - DNA, 23 bp, Artificial Sequence
  Description of Artificial Sequence Synthetic oligonucleotide
ctctttgctgccatttctggaat

SEQ ID NO: 20 - DNA, 20 bp, Artificial Sequence
  Description of Artificial Sequence Synthetic oligonucleotide
tctattggtgaagattcctg

(19) United States
(12) Patent Application Publication
(10) Pub. No.: US 2008/0299546 A1
(43) Pub. Date: Dec. 4, 2008

(54) Canine Cd20 Gene

(75) Inventors: Rui Kano, Tokyo (JP); Atsuhiko Hasegawa, Tokyo (JP); Chika Inoue, Kanagawa (JP)

Correspondence Address: YOUNG & THOMPSON, 209 Madison Street, Suite 500, ALEXANDRIA, VA, 22314, US

(73) Assignee: NIHON UNIVERSITY, Chiyoda-ku, Tokyo (JP)

(21) Appl. No.: 10/588,903

(22) PCT Filed: Feb. 9, 2005
(86) PCT No.: PCT/JP2005/001880

§ 371 Date: Sep. 12, 2006

(30) Foreign Application Priority Data
Feb. 10, 2004 (JP) 2004-033810

(51) Int. Cl. C12Q 1/68; C07K 14/00; C12N 15/11; C12N 15/00; C12N 5/06; C07H 21/04

(52) U.S. Cl. 435/6; 530/350; 536/23.5; 435/320.1; 435/325; 536/24.33

(57) ABSTRACT

It is intended to clarify the CD20 amino acid sequence and its gene sequence which are essentially required in constructing an anti-CD20 antibody useful in treating animal malignant lymphoma. It is also intended to provide a method of diagnosing canine malignant lymphoma by using the gene sequence. Using monocytes in canine blood as a sample, mRNA is obtained and the full base sequence of canine CD20 gene (SEQ ID NO:2) is determined. Based on this sequence, its amino acid sequence (SEQ ID NO:1) is determined. Comparing the homologies with human and mouse CD20 genes and amino acid sequences, it is identified as canine CD20 gene. Moreover, a primer specific to the canine gene is constructed and the expression of the CD20 gene in a sample is examined, thereby giving a method of diagnosing canine B lymphocyte-origin malignant lymphoma.

### TECHNICAL FIELD

The present invention relates to canine CD20 gene to be used for development of an antibody therapy, diagnosis, or the like for malignant lymphoma. Moreover, the present invention relates to a method of diagnosing canine B lymphocyte-origin malignant lymphoma by amplifying the canine CD20 gene to examine expression of the canine CD20 gene.

### BACKGROUND ART

Malignant lymphoma is caused by tumorigenesis in lymphatic tissues in a living body. Human malignant lymphomas are classified into Hodgkin's lymphomas and non-Hodgkin's lymphomas, and the non-Hodgkin's lymphomas are classified into T lymphocyte-origin, NK cell-origin, and B lymphocyte-origin lymphomas depending on its origin. They are divided into low-grade malignant lymphoma, intermediate-grade malignant lymphoma, and high-grade malignant lymphoma depending on the rates of malignant progression.

In Japanese patients, most malignant lymphomas are non-Hodgkin's lymphomas. Although the lymphomas often occur in the lymph nodes, they may occur in the entire body where lymphatic tissues are present, such as skin, brain, eye, nasal cavity, paranasal cavity, tonsil, pharynx, salivary gland, thyroid gland, mammary gland, lung, mediastinum, pleura, stomach, small intestine, large intestine, liver, spleen, testis, ovarium, and bone, and the symptoms vary depending on the tissues.

Malignant lymphoma is one of tumors that often occur in dogs or the like, and in the case of dogs, multicentric lymphoma often occurs and causes swollen lymph nodes in the entire body. If the multicentric lymphoma becomes malignant, it spreads through lymphatic tissues to the lung, liver, spleen, or bone marrow, resulting in symptoms such as jaundice and anemia.

Meanwhile, the lymphoma also includes thymic lymphoma that causes accumulation of water in the chest cavity due to swollen lymph nodes of a thymus gland, digestive lymphoma that causes tumorigenesis in lymphatic tissues of a digestive organ, and the like. Such malignant lymphomas progress rapidly, and if a diseased animal such as a dog receives no treatment, it will die about 100 days after discovery on average. Therefore, early detection and appropriate treatments after the detection are desired.

Therapies for malignant lymphoma include a plurality of therapies such as radiation therapy, chemotherapy, and surgical therapy; conventionally, the radiation therapy and chemotherapy have been mainly performed.

The radiation therapy is often used in the case that there are several lesion sites of malignant lymphoma. It is a therapy to kill the lesion sites with pinpoint accuracy by irradiating a radiation ray to tumor cells and is the most suitable therapy for removing early tumors, but it has side effects such as skin damage, mucosal damage, and lung damage.

The chemotherapy is often used in the case that tumor cells spread to the entire body such as lymphatic tissues, organs, bone marrow, and blood. It is a therapy to kill the lesion sites in the entire body by administering anticancer drugs that has cytotoxicity against the tumor cells and is a unique systemic therapy for malignant lymphoma.

For human malignant lymphomas, CHOP therapy using anticancer drugs including cyclophosphamide, adriamycin, vincristine, and prednisolone in combination is normally performed. Meanwhile, for canine malignant lymphomas, COAP protocol using anticancer drugs including cyclophosphamide, vincristine, cytosine arabinoside, and prednisolone in combination is performed (Non-Patent Document 1).

Such anticancer drugs are cytotoxins that disrupt all cells and may affect normal cells, but they are used by taking advantage of the nature that tumor cells is damaged more easily because the tumor cells grow more rapidly than normal cells.

However, the anticancer drugs cause damage to normal cells that divide rapidly, e.g., to blood cells, hair root cells, gastrointestinal cells, germ cells, or the like as well as tumor cells, so that side effects such as decrease in the number of white blood cells, hair loss, and nausea are caused.

Although the radiation therapy, chemotherapy, and the like are therapies that are now mainly used, they may cause strong side effects on a living body, and even if the treatments result in remission but then lymphomas recur in many cases, so that they are not necessarily considered as therapies that lead to complete recovery.

Meanwhile, considerable expense is required to perform the therapies. According to USA statistics, it costs about 1,500 to 1,800 dollars to treat not only humans but also dogs (30 to 32 kg), so that the therapies are not necessarily considered as inexpensive therapies. Therefore, to solve such problems, there is required a therapy for malignant lymphoma that is safer, leads to complete recovery, and may be performed inexpensively.

The chemotherapy is considered to have a problem in that an anticancer drug to be used is a cytoxin that attacks all cells. Therefore, monoclonal antibody therapy that may specifically attack only target tumor cells has gotten attention recently (Non-Patent Document 2).

It is a therapy using an antigen that is specifically expressed in target tumor cells and using an antibody specific to the antigen to specify the tumor cells, thereby concentrating a drug on tumor cells.

The therapy for human malignant lymphoma includes a method using a CD20 antigen that is specifically expressed in the surfaces of B lymphocytic cells and is expressed at a high level in malignant lymphoma and using an anti-CD20 antibody that specifically recognizes the antigen.

When the anti-CD20 antibody binds to the CD20 antigen in a tumor cell, an antibody or complement-mediated immunoreaction occurs, thereby causing damage to the tumor cell. The action mechanism is different from that of a conventional anticancer drug, and the antibody has an effect on only a target tumor cell, so that it has no influence on hematopoietic stem cells or the like where the CD20 antigen is not expressed on the surface. In fact, it is known that side effects are only hypersensitivity or allergy-like symptoms, and hair loss, nausea, and the like as induced by an anticancer drug are hardly caused.

The anti-CD20 antibody has been prepared as a chimeric mouse/human antibody derived from human and has been covered by insurance for CD20-positive low-grade or follicular B-cell non-Hodgkin's lymphomas since 2001 in Japan. It is marketed by Zenyaku Kogyo as a drug under the trade name of rituxan and generic name of rituximab.

Such antibody therapy, which has excellent therapeutic effects and causes fewer side effects, is expected as a therapy that revolutionizes therapies for malignant lymphoma.

Although such useful therapy for human has been established, the antibody therapy has never been performed for diseased animals such as dogs. However, there are many animals suffering from malignant lymphoma, so that it has been strongly desired to establish the antigen therapy. Therefore, the inventors of the present invention have clarified in the present invention a sequence of canine CD20 gene that is considered to be essential for establishing the antibody therapy. The sequence of canine CD20 gene clarified in the present invention is essential for producing a canine anti-CD20 antibody and may be used for development of an antibody therapy, diagnosis, or the like of canine malignant lymphoma.

In addition, the sequence of canine CD20 gene clarified in the present invention can be used for diagnosis of canine malignant lymphoma such that the sequence is used for producing a primer that may specifically amplify canine CD20 gene to examine expression of the canine CD20 gene.

Non-patent Document 1: Feline and Canine Lymphoma, Atsuhiko Hasegawa, Hajime Tsujimoto, translation supervisor: Small Animal Internal Medicine, 1127-1137.
Non-Patent Document 2: Accommodation guideline for hematopoietic stem cell transplantation, The Japan Society for Hematopoietic cell transplantation, April 2002.

### DISCLOSURE OF THE INVENTION

### Problems to be Solved by the Invention

An object of the present invention is to clarify a sequence of canine CD20 gene essential for the production of canine anti-CD20 antibody. A further object of the present invention is to provide a method of diagnosing canine malignant lymphoma by producing a primer that may specifically amplify the canine CD20 gene using the clarified sequence of the canine CD20 gene to examine expression of the canine CD20 gene.

### Means for Solving the Problems

The inventors of the present invention have made extensive studies on the above problems. As a result, they have isolated genes using mononuclear cells in canine blood as samples and have purified canine CD20 gene, thereby clarifying the sequence. The canine CD20 gene was found to have high homology of 81.0% with human CD20 gene and to have homology of 71.8% with mouse CD20 gene. Meanwhile, CD20 amino acid sequence encoded by such gene sequence was found to have high homology of 72.8% with human CD20 amino acid sequence and to have homology of 68.2% with mouse CD20 amino acid sequence.

Meanwhile, CD20 is expressed outside a cell membrane and is recognized as an antigen, so that the inventors have examined homology between the amino acid sequence in an extramembrane region of the CD20 clarified in the present invention and an amino acid sequence in an extramembrane region of the human CD20, and the homology was found to be 66.6%. The results suggested that the canine CD20 gene is useful for production of an anti-CD20 antibody.

Therefore, it is considered that a sequence having a homology of 70% or higher or 80% or higher with the amino acid sequence, DNA sequence, or RNA sequence clarified by the present invention is also effective, and the sequence also includes one in which one or more amino acids or bases are deleted, substituted, added, or inserted as long as it is within such range.

Moreover, the inventors of the present invention have found out that the expression of canine CD20 gene may be examined by producing a primer that may specifically amplify the canine CD20 gene using the sequence of the canine CD20 gene clarified in the present invention to amplify the canine CD20 gene in a sample, thereby completing a method of diagnosing canine malignant lymphoma.

That is, the present invention relates to:

(1) a canine CD20 protein having an amino acid sequence according to SEQ ID NO: 1;

(2) a protein having a homology of 70% or higher with an amino acid sequence according to SEQ ID NO: 1;

(3) a protein having a homology of 80% or higher with an amino acid sequence according to SEQ ID NO: 1;

(4) a DNA encoding canine CD20 according to SEQ ID NO: 2;

(5) a polynucleotide having a homology of 70% or higher with a DNA sequence according to SEQ ID NO: 2;

(6) a polynucleotide having a homology of 80% or higher with a DNA sequence according to SEQ ID NO: 2;

(7) an RNA encoding canine CD20 according to SEQ ID NO: 3;

(8) a polynucleotide having a homology of 70% or higher with an RNA sequence according to SEQ ID NO: 3;

(9) a polynucleotide having a homology of 80% or higher with an RNA sequence according to SEQ ID NO: 3;

(10) a plasmid vector comprising a canine CD20 gene fragment according to the item (4);

(11) a plasmid vector comprising a polynucleotide according to the item (5) or (6);

(12) a plasmid vector comprising a canine CD20 gene fragment according to the item (7);

(13) a plasmid vector comprising a polynucleotide according to the item (8) or (9);

(14) a transformant comprising a plasmid according to the item (10) or (11);

(15) a transformant comprising a plasmid according to the item (12) or (13);

(16) a primer according to SEQ ID NO:19 for amplifying canine CD20 gene or a fragment thereof;

(17) a primer according to SEQ ID NO: 20 for amplifying canine CD20 gene or a fragment thereof; and

(18) a method of diagnosing canine malignant lymphoma by amplifying canine CD20 gene or a fragment thereof to examine expression of the canine CD20 gene using a primer according to the item (16) or (17).

### Effect of the Invention

The sequence of canine CD20 gene clarified by the present invention is essential for the production of a canine anti-CD20 antibody. The sequence of canine CD20 gene of the present invention may be used for development of an antibody therapy, diagnosis, or the like of canine malignant lymphoma. Moreover, canine malignant lymphoma may be diagnosed in such a manner that a primer that may specifically amplify canine CD20 gene is produced using the sequence of the canine CD20 gene clarified in the present invention to amplify the canine CD20 gene in a sample, thereby examining the expression of the canine CD20 gene.

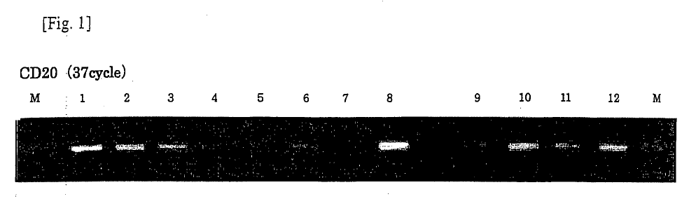

### BRIEF DESCRIPTION OF DRAWING

[FIG. 1]

The FIGURE shows the expressions of canine CD20 genes in samples (Example 8). The symbol M in the FIGURE represents a marker, and the numerals 1 to 10 correspond to the samples 1 to 10 in Table 1. The numerals 11 and 12 represent samples from normal canine lymph nodes.

### BEST MODE FOR CARRYING OUT THE INVENTION

In determination of a sequence of CD20 gene using PCR, which is an object of the present invention, sample accuracy and designs of primers to be used are important. Extraction of mRNA or the like may be performed using a commercially-available kit.

In addition, in the case of producing a primer that may specifically amplify canine CD20 gene or a fragment thereof using the sequence of the canine CD20 gene clarified in the present invention, the primer to be produced may be DNA or RNA as long as it may be a gene that may examine the expression of the canine CD20 gene or the fragment thereof, and in particular, a cDNA synthesized from mRNA is easy to handle and preferable. The fragment of the canine CD20 gene for examining the expression preferably has a sequence containing a gene in an extramembrane region of the canine CD20.

Hereinafter, examples that may embody the present invention are shown but should not be construed as limiting the present invention in any way.

### EXAMPLE 1

### Preparation of Sample

### (1) Separation of Mononuclear Cell

Blood collected from a normal dog (5 ml) was subjected to an anticoagulation treatment with heparin, and heparinized saline (5 ml) was added, followed by tumble mixing (total volume 10 ml). To a centrifugation tube was poured 5 ml of Lymphoprep, and a sample was layered thereon, followed by centrifugation at room temperature at 800×g using a centrifugal machine, thereby separating mononuclear cells. The mononuclear cells were separated using Lymphoprep (Axis-Shield Pos AS).

### (2) RNA Extraction

To the resultant mononuclear cells was added Buffer RLT (supplemented with “-mercaptoethanol) containing a guanidine salt to lyse the cells. The resultant mixture was added to a QIAshredder spin column, and then centrifugation was performed at 1,000×g for 2 minutes, followed by homogenization (QIAshredder, QIAGEN). 70%-Ethanol was added, and the mixture was mixed using a pipette and applied to an RNeasy mini spin column, followed by centrifugation at 8,000×g for 15 seconds to discard flow-through. RNA was adsorbed on a silica gel membrane of the RNeasy mini spin column.

Subsequently, Buffer RW1 was added to the RNeasy column, and incubation was performed at room temperature for 5 minutes, followed by centrifugation at 8,000×g for 15 seconds to discard flow-through. Buffer RPE was applied to the RNeasy column, and then centrifugation was performed at 8,000×g for 15 seconds. The procedures were repeated twice to remove impurities.

Finally, an appropriate volume of RNase free water was added, and centrifugation was performed at 8,000×g for 1 minute to elute RNA. The RNA was extracted using a kit (RNeasy Mini Kit, QIAGEN).

### (3) DNase Treatment

To prevent a minute quantity of genomic DNA from being mixed into the resultant RNA, genomic DNA was removed by the following DNase treatment.

Buffer and DNaseI supplied with the kit were added to the RNA solution, and the mixture was incubated at 37° C. for 30 minutes. DNase Inactivation Reagent was further mixed, and the mixture was incubated at room temperature for 2 minutes. Centrifugation was performed at 8,000×g for 1 minute, and the supernatant was transferred to another new tube, thereby producing a DNA-free RNA solution. The DNase treatment was performed using a kit (DNA-free, Ambion).

(4) Synthesis of cDNA

To the RNA solution treated with a DNase were added 10× Buffer RT, dNTP Mix, RT (reverse transcriptase), Ribonuclease Inhibitor cloned (Invitrogen), and an oligo dT primer having a sequence of SEQ ID NO: 6 (Proligo Japan KK.), and the mixture was incubated at 37° C. for 1 hour, thereby synthesizing a cDNA. The cDNA was synthesized using a kit (Omniscript, QIAGEN).

### EXAMPLE 2

### Cloning of Canine CD20 Gene Fragment

### (1) PCR

A sense primer having a sequence of SEQ ID NO: 7 and a reverse primer having a sequence of SEQ ID NO: 8 were designed from a region with high homology on the basis of base sequences of human/mouse CD20 genes, and PCR was performed using the synthesized cDNA as a template (TaKaRa Taq, TaKaRa).

A reaction solution supplemented with a thermostable DNA polymerase (rTaq) supplied with the kit was subjected to heat denaturation at 94° C. for 5 minutes, followed by 35 cycles of PCR under conditions of 94° C. for 1 minute, 53 to 60° C. for 1 minute, and 72° C. for 2 minutes to amplify a CD20 gene fragment. Subsequently, the PCR products were confirmed by agarose gel electrophoresis.

### (2) TA Cloning/Transformation

The resultant CD20 gene fragment was integrated into a plasmid vector (pCR vector), and the vector was transformed with Escherichia coli (TA Cloning Kit, Invitrogen), followed by proliferation of Escherichia coli in LB medium supplemented with ampicillin/X-gal.

The boiling method confirmed that the proliferated Escherichia coli has a plasmid DNA including an intended gene fragment. The plasmid was extracted form the Escherichia coli using a kit (BioRad plasmid kit, BioRad).

### EXAMPLE 3

### Determination of Base Sequence of Canine CD20 Gene Fragment

### Sequencing

A cycle sequencing reaction was performed using the resultant plasmid as a template and using an M13 forward (−21) primer having a sequence of SEQ ID No: 9 and an M13 reverse primer having a sequence of SEQ ID No: 10, which are specific to the used pCR vector. The reaction was performed using a thermal cycler under conditions consisting of 25 cycles of 96° C. for 10 seconds, 50° C. for 5 seconds, and 60° C. for 4 minutes. The reaction solution was prepared using a kit (Big Dye Terminator v3.1 Cycle Sequencing Kit, Applied Biosystems). In the used Terminator Ready Reaction Mix supplied with the kit, a DNA polymerase and dideoxyribonucleoside triphosphate (ddNTP) labeled with a fluorescent dye had previously been mixed.

After completion of the reaction, the sequencing products were purified by ethanol/EDTA precipitation to remove unreacted fluorescent substances. The resultant products were dissolved in a Template Suppression Reagent (TSR), and base sequences were determined using a sequencer (ABI PRISM 310 Genetic Analyzer, Applied Biosystems).

### EXAMPLE 4

### Determination of Base Sequence of Entire Canine CD20 Gene

To determine the full length of cDNA of the CD20 gene, novel primers were designed using the partially determined base sequences, followed by 5′- and 3′-RACE PCR (5′RACE System of Rapid Amplification of cDNA Ends version 2.0/3′RACE System of Rapid Amplification of cDNA Ends, Invitrogen).

### (1) 3′RACE Method

1) Synthesis of cDNA

An adaptor primer having a sequence of SEQ ID NO: 11 was mixed in the RNA solution, and incubation was performed at 70° C. for 10 minutes. The mixture was placed on ice for 3 minutes, and Buffer/0.1 M DTT/10 mM dNTP was added and mixed, followed by heating at 37° C. for 2 minutes. Subsequently, a reverse transcriptase (SuperScript II Reverse Transcriptase) was added and mixed, followed by a reaction at 42° C. for 1 hour, thereby synthesizing a cDNA.

### 2) PCR

A reaction solution supplemented with a thermostable DNA polymerase (rTaq) was subjected to heat denaturation at 94° C. for 5 minutes, followed by 35 cycles of first PCR under conditions of 94° C. for 1 minute, 54° C. for 1 minute, and 72° C. for 2 minutes using a sense primer having a sequence of SEQ ID NO: 12 (GSP1) and a reverse primer having a sequence of SEQ ID NO: 13 (Universal Amplification Primer: supplied with the kit). Subsequently, a reaction solution including the first PCR product as a template was prepared and subjected to heat denaturation at 94° C. for 5 minutes, followed by 35 cycles of nested PCR (second PCR) under conditions of 94° C. for 1 minute, 55° C. for 1 minute, and 72° C. for 2 minutes using a sense primer having a sequence of SEQ ID NO: 14 (GSP2) and a reverse primer having a sequence of SEQ ID NO: 13 (Universal Amplification Primer: supplied with the kit), thereby amplifying a CD20 gene fragment.

The used reagents except rTaq (TaKaRa Taq, TaKaRa) were supplied with the kit for 3′RACE method. Subsequently, PCR products were confirmed by means of agarose gel electrophoresis.

### (2) 5′RACE Method

1) Synthesis of cDNA

A sense primer having a sequence of SEQ ID NO: 15 (GSP1) was added to the RNA solution, and the mixture was incubated at 70° C. for 10 minutes. The mixture was placed on ice for 1 minute, and then Buffer, MgCl₂, DTT, dNTP, and a reverse transcriptase (SuperScript II Reverse Transcriptase) were added, followed by incubation at 42° C. for 1 hour. The mixture was heated at 70° C. for 15 minutes to deactivate the Reverse Transcriptase. RNase Mix was added and mixed, and the mixture was heated at 37° C. for 30 minutes to degrade RNA.

### 2) Purification of DNA

A binding solution was added to the cDNA reaction solution, and the mixture was mixed and transferred to a spin cartridge. The mixture was centrifuged at 1,000×g for 2 minutes to discard flow-through. Wash Buffer was added, and the mixture was centrifuged at 1,000×g for 2 minutes to discard flow-through. The spin cartridge was transferred to another new tube, and sterilized water with a temperature of 65° C. was added. The mixture was incubated at room temperature for 1 minute, followed by centrifugation at 14,000 rpm for 1 minute to collect the solution.

### 3) Addition of Homopolymer Using TdT

Sterilized water 7.5, Buffer, MgCl₂, dCTP, and cDNA were mixed, and the mixture was heated at 94° C. for 1 to 2 minutes. The mixture was placed on ice for 1 minute, and TdT was added and mixed, followed by incubation at 37° C. for 10 minutes and at 65° C. for 10 minutes.

### 4) PCR

A reaction solution supplemented with a thermostable DNA polymerase (rTaq) was subjected to heat denaturation at 94° C. for 5 minutes, followed by 35 cycles of first PCR under conditions of 94° C. for 1 minute, 50° C. for 1 minute, and 72° C. for 2 minutes using a sense primer having a sequence of SEQ ID NO: 16 (GSP2) and a reverse primer having a sequence of SEQ ID NO: 17 in sequence table (Anchor Primer: supplied with the kit). Subsequently, a reaction solution including the first PCR product as a template was prepared and subjected to heat denaturation at 94° C. for 5 minutes, followed by 35 cycles of nested PCR (second PCR) under conditions of 94° C. for 1 minute, 58° C. for 1 minute, and 72° C. for 2 minutes using a sense primer having a sequence of SEQ ID NO: 18 (GSP3) and a reverse primer having a sequence of SEQ ID NO: 13 (Universal Amplification Primer: supplied with the kit), thereby amplifying a CD20 gene fragment. The used reagents except rTaq (TaKaRa Taq, TaKaRa) were supplied with the kit for 3′RACE method. Subsequently, PCR products were confirmed by means of agarose gel electrophoresis.

### EXAMPLE 5

### Method of Identifying Canine CD20 Gene

The homology between the analyzed base sequence of the gene and those of human/mouse genes was confirmed. The gene was found to have high homologies of 81.0% with human CD20 genome (exon) registered with Genbank and 71.8% with mouse CD20 mRNA, so that the analyzed gene was identified as canine CD20. The DNA sequence of the canine CD20 is shown in SEQ ID NO: 3, while the RNA sequence of the canine CD20 is shown in SEQ ID NO: 4.

### EXAMPLE 6

### Method of Identifying Amino Acid Sequence

The amino acid sequence of the CD20 was identified using the analyzed base sequence of the gene. The identified amino acid sequence of the canine CD20 is shown in SEQ ID NO: 1.

The homology between the amino acid sequence and those of human/mouse genes was confirmed. The gene was found to have high homologies of 72.8% with the amino acid sequence of a human CD20 registered with Genbank and 68.2% with a mouse, so that the analyzed gene was identified as canine CD20.

Meanwhile, an amino acid sequence of an extramembrane region in the human CD20 had homology of 66.6% with an amino acid sequence of canine CD20 that was considered to correspond with the sequence. The sequence of the extramembrane region in the CD20 is shown in SEQ ID NO: 2, while the DNA sequence is shown in SEQ ID NO: 5.

### EXAMPLE 7

### Preparation of Primer for Amplifying Canine CD20 Gene

From the thus-determined base sequence of the entire canine CD20 gene, a sequence suitable as a primer to amplify a 506-bp sequence that contains a gene of the extramembrane region in the canine CD20 according to SEQ ID NO: 5 was selected, and a sense primer having a sequence of SEQ ID NO: 19 and a reverse primer having a sequence of SEQ ID NO: 20 were designed.

### EXAMPLE 8

### Method of Diagnosing Canine Malignant Lymphoma

### (1) Preparation of Sample

From tumor cells collected from normal canine lymph nodes (2 subjects) and lymph nodes affected by lymphoma (Stage III: 1 subject, IV: 1 subject, V: 8 subjects), total RNAs were extracted using a kit (Rneasy Mini Kit, QIAGEN). Subsequently, contaminating DNAs were removed using a kit (DNA free kit, Ambion), and then template cDNAs to be used for PCR reactions were synthesized from the total RNAs (2″g) using a kit (Omniscript RT kit, QIAGEN). Note that the origin of each of the lymphoma samples (10 subjects) was determined by surface antigen analysis, and the origin and properties of each sample are shown in Table 1.

To equalize the levels of template cDNAs in the samples, the expression levels of canine GAPDH gene levels were determined as a control gene for gene expression, and cDNA levels in the samples were equalized on the basis of the levels. For canine GADPH gene in each sample, a reaction solution supplemented with primers specific to the GADPH gene (sense primer 5′-CTCTTTGCTGCCATTTCTGGAAT-3, reverse primer 5′-TCTATTGGTGAAGATTCCTG-3′) and a thermostable DNA polymerase (rTaq) was subjected to heat denaturation at 94° C. for 5 minutes, followed by 26 cycles of PCR under conditions of 94° C. for 30 seconds, 55° C. for 30 seconds, and 72° C. for 1 minute (elongation reaction 7 minutes) (TaKaRa Taq, TaKaRa). The genes amplified by the PCR reactions were electrophoresed on a 2%-agarose gel, and the gel was stained with ethidium bromide and then observed under irradiation with an ultraviolet ray.

TABLE 1

| | Breeds and ages of | Anatomical | Cell surface | | Therapeutic |
|---|---|---|---|---|---|
| No. | dogs | classification | antigen | Stage | process |
| 1 | Golden Retriever, | Multicentric form | B cell | Vb | Death after two |
| | 7 years old | | | | recurrences |
| 2 | Shih Tzu, | Multicentric form | B cell | Vb | Death after two |
| | 13 years old | | | | recurrences |
| 3 | Maltese, 6 years old | Multicentric form | B cell | Va | Unknown |
| 4 | Golden Retriever, | Multicentric form | T cell | Vb | Anticancer drug |
| | 10 years old | | | | resistance |
| 5 | Siberian Husky, | Multicentric form | Unknown | IIIb | Three recurrences |
| | 11 years old | | | | |
| 6 | Golden Retriever, | Multicentric form | B cell | Vb | Death after three |
| | 7 years old | | | | recurrences |
| 7 | Labrador Retriever, | Mediastinal form | nonB nonT cell | Vb | Death on the 3rd |
| | 3 years old | | | | day of illness |
| 8 | Golden Retriever, | Multicentric form | B cell | Vb | Death after two |
| | 4 years old | | | | recurrences |
| 9 | Kind unknown, | Multicentric form | B cell | IVa | Unknown |
| | 7 years old | | | | |
| 10 | Golden Retriever, | Multicentric form | B cell | Va | Unknown |
| | 8 years old | | | | |

### (2) Amplification of Sample

The levels of cDNAs in the samples were equalized to be used as templates, and a reaction solution supplemented with: a sense primer for amplifying a CD20 gene having a sequence of SEQ ID NO: 19 or a fragment thereof; a reverse primer for amplifying a CD20 gene having a sequence of SEQ ID NO: 20 or a fragment thereof; and a thermostable DNA polymerase (rtaq) was subjected to heat denaturation at 94° C. for 5 minutes, followed by 37 cycles of PCR under conditions of 94° C. for 30 seconds, 55° C. for 30 seconds, and 72° C. for 1 minute (elongation reaction 7 minutes) (TaKaRa Taq, TaKaRa). The cDNAs in the samples amplified by the PCR reactions were electrophoresed on an agarose gel, and the gel was stained with ethidium bromide and then observed under irradiation with an ultraviolet ray. Consequently, expressions of canine CD20 genes were determined from the results of presence or absence of amplified fragments containing genes of extramembrane regions in the canine CD20 in samples, and the expressions are used for diagnosis of canine B lymphocyte-origin malignant lymphoma. The expressions of canine CD20 genes in the samples are shown in FIG. 1.

### (3) Results

As shown in FIG. 1, in the cases of 2 subjects of normal canine lymph nodes (11 and 12 in FIG. 1) and 7 subjects of B lymphocyte-origin malignant lymphoma (1 to 3, 6, and 8 to 10 in FIG. 1), expressions of CD20 genes were observed, while in the cases of one subject of T lymphocyte-origin malignant lymphoma (4 in FIG. 1), one subject of undifferentiated lymphoma cell (7 in FIG. 1), and unknown one subject (5 in FIG. 1), expressions of CD20 genes were not observed. The results revealed that canine B lymphocyte-origin malignant lymphoma may be diagnosed such that the expression of CD20 gene is examined by using primers for amplifying the CD20 genes of SEQ ID NOS: 19 and 20 of the present invention.

### INDUSTRIAL APPLICABILITY

The sequence of canine CD gene revealed by the present invention is useful for development of an antibody therapy, diagnosis, or the like of canine malignant lymphoma. More specifically, the gene sequence may help produce a canine anti-CD20 antibody and develop novel therapeutic or diagnostic agents for canine malignant lymphoma or apparatus using them.

1-18. (canceled)

19. A canine CD20 protein having an amino acid sequence according to SEQ ID NO: 1.

20. A protein having a homology of 70% or higher with an amino acid sequence according to SEQ ID NO: 1, and having the same function as a canine CD20 protein.

21. A protein having a homology of 80% or higher with an amino acid sequence according to SEQ ID NO: 1, and having the same function as a canine CD20 protein.

22. A DNA encoding canine CD20 according to SEQ ID NO: 3.

23. A polynucleotide having a homology of 70% or higher with a DNA sequence according to SEQ ID NO: 3.

24. A polynucleotide having a homology of 80% or higher with a DNA sequence according to SEQ ID NO: 3.

25. An RNA encoding canine CD20 according to SEQ ID NO: 4.

26. A polynucleotide having a homology of 70% or higher with an RNA sequence according to SEQ ID NO 4.

27. A polynucleotide having a homology of 80% or higher with an RNA sequence according to SEQ ID NO: 4.

28. A plasmid vector comprising a canine CD20 gene fragment according to claim 22.

29. A plasmid vector comprising a polynucleotide according to claim 23.

30. A plasmid vector comprising a canine CD20 gene fragment according to claim 25.

31. A plasmid vector comprising a polynucleotide according to claim 26.

32. A transformant comprising a plasmid vector according to claim 28.

33. A transformant comprising a plasmid vector according to claim 30.

34. A primer according to SEQ ID NO: 19 for diagnosing canine malignant lymphoma which amplifies canine CD20 gene or a fragment thereof.

35. A primer according to SEQ ID NO: 20 for diagnosing canine malignant lymphoma which amplifies canine CD20 gene or a fragment thereof.

36. A method of diagnosing canine malignant lymphoma by amplifying canine CD20 gene or a fragment thereof to examine expression of the canine CD20 gene using a primer according to claim 34.

37. A plasmid vector comprising a polynucleotide according to claim 24.

38. A plasmid vector comprising a polynucleotide according to claim 27.

39. A transformant comprising a plasmid vector according to claim 29.

40. A transformant comprising a plasmid vector according to claim 31.
